(19) United States
(12) Patent Application Publication
(10) Pub. No.: US 2025/0152421 A1
(43) Pub. Date: May 15, 2025

(54) CORNEAL PATH ONE-WAY AQUEOUS HUMOR DRAINAGE DEVICE

(71) Applicants: BEIJING TONGREN HOSPITAL, CAPITAL MEDICAL UNIVERSITY, Beijing (CN); BEIJING INSTITUTE OF OPHTHALMOLOGY, Beijing (CN)

(72) Inventors: Ningli WANG, Beijing (CN); Yingyan MAO, Beijing (CN); Yi ZHEN, Beijing (CN); Lihua DU, Beijing (CN); Chen XIN, Beijing (CN); Wei ZHANG, Beijing (CN); Jing SHEN, Beijing (CN)

(73) Assignees: BEIJING TONGREN HOSPITAL, CAPITAL MEDICAL UNIVERSITY, Beijing (CN); BEIJING INSTITUTE OF OPHTHALMOLOGY, Beijing (CN)

(21) Appl. No.: 18/839,013

(22) PCT Filed: Dec. 12, 2022
(86) PCT No.: PCT/CN2022/138314

§ 371 Date: Aug. 15, 2024

(30) Foreign Application Priority Data
Feb. 16, 2022 (CN) 202210143155.5
Sep. 29, 2022 (CN) 202211198306.3

(51) Int. Cl. A61F 9/007

(52) U.S. Cl. CPC A61F 9/00781; A61F 2240/001

(57) ABSTRACT

The invention provides a corneal path one-way aqueous humor drainage device, in which a Tesla valve unit group drainage channel is formed for draining aqueous humor to the ocular surface, and the drainage channel is a one-way drainage. The aqueous humor diverter provided by the utility model introduces a Tesla valve as a structure to prevent the backflow of aqueous humor. When the liquid flows into the Tesla valve unit group from the liquid outlet, the Tesla valve unit group can block the liquid from entering the liquid. Oral flow. The aqueous humor drainage device can be used for glaucoma patients to reduce intraocular pressure and can also be used for severe dry eye; it is made of materials with good biocompatibility and has high clinical drainage efficiency; its structure.

### TECHNICAL FIELDS

The present invention relates to the technical field of ophthalmic medical devices, and in particular relates to a corneal path unidirectional aqueous drainage device.

### BACKGROUND TECHNOLOGY

Glaucoma is the second most irreversible blinding eye disease in the world. According to statistics, there are about 60.5 million glaucoma patients in the world, 8.4 million are blind in both eyes, and the direct cost of glaucoma treatment in the U.S. reaches 2.9 billion dollars every year. There are no statistics on the cost of glaucoma treatment in China, but since the number of glaucoma patients in China is 5-6 times higher than that of the United States, it is estimated that the direct cost of glaucoma treatment in China can reach 15-18 billion dollars if we treat all glaucoma patients, and the indirect economic losses caused by glaucoma may be even greater. Worse still, with the aging of the population, by 2020, the number of glaucoma patients will have increased to 79.64 million, and 11.2 million people may become blind in both eyes due to glaucoma. 2022 is the first time that the 14th Five-Year Plan proposes to strengthen the prevention and treatment of key eye diseases in key populations, and explicitly points out that it is necessary to improve the ability of early diagnosis and treatment of glaucoma, which is the most common cause of glaucoma in China. With the accelerated aging of the population and the rise of national health awareness, the future glaucoma diagnosis and treatment market has huge growth potential. Therefore, research on the prevention and treatment of glaucoma is of great social and economic significance.

At the present stage, the main means of clinical treatment for glaucoma in China are medication and surgery. In the early stage, most of the patients are treated with medication (eye drops), while in the later stage, they are treated with the “gold standard” surgery “trabeculectomy”, which was started in 1968. Currently, all treatments are aimed at controlling intraocular pressure (IOP) and preserving vision in a rational and effective manner throughout the patient's lifespan. The above two therapeutic interventions have been in use for many years, but the drug treatment may have disadvantages such as drug drift, poor patient compliance, lifelong use of drugs, adverse drug reactions, etc.; for glaucoma surgery, the “gold standard” trabeculectomy, there is a long learning curve for the doctor, a large amount of surgical trauma, large destruction of the normal eye structure and function, and complex complications. Trabeculectomy, the “gold standard” of glaucoma surgery, has a long learning curve for the surgeon, greater surgical trauma, greater damage to the normal structure and function of the eye, and complicated complications.

In response to these pain points, experts, scholars, and companies in this field have been exploring and trying to find alternative surgical methods with lower complication rates and less trauma. In recent years, the new generation of MIGS surgery, which has become popular internationally and has been verified by more than one million people, is attracting more and more attention. With its advantages of small surgical incision, short operation time, fast postoperative recovery, and low doctor learning curve, it has attracted more and more attention from ophthalmologists. It is understood that the new generation of MIGS, as an emerging method and popular trend in glaucoma treatment, has been widely used internationally. In the four years since 2018, more than one million glaucoma patients around the world have undergone MIGS surgical intervention and treatment.

Based on the principle of reducing intraocular pressure (IOP), the current mainstream MIGS procedures and products can be divided into three types: (1) increasing aqueous humor outflow through the trabecular meshwork; (2) suprachoroidal aqueous humor drainage; (3) Subconjunctival aqueous humor drainage. Among them, the aqueous humor drainage of (1) and (2) MIGS surgical products mainly relies on the original physiological aqueous humor outflow channel, and its intraocular pressure reduction range is smaller than that of traditional trabeculectomy. It is suitable for patients with early and middle-stage open-angle glaucoma. The advantages are: Little impact on conjunctiva. (3) The mechanism of action of MIGS surgical products is similar to that of traditional trabeculectomy, and the magnitude of intraocular pressure reduction is also similar. The advantage is that the incidence of postoperative complications is lower than that of trabeculectomy.

Among the currently available MIGS surgeries, the one that utilizes trabecular meshwork schlemm's tube access is: iStent, which was approved by the FDA in 2012 and is suitable for patients with glaucoma with a mild course of disease. Its 1-year effective rate is 33.7%, but its disadvantage is the filtration channel. Easy to block. The ones that use the ciliary body suprachoroidal access include iStent inject W, which was approved by the FDA in 2020, and there are currently no relevant literature reports; Hydrus, which was approved by the FDA in 2018, with a 1-year effective rate of 37.1%; iStent inject, which was approved by CE in 2010, the 2-year effective rate is 31.0%; CyPass Micro-Stent was withdrawn from the market in 2018, with the disadvantages of high risk of hypotonia and filtration channel obstruction; SOLX gold shunt, launched in Canada, with a 5-year effective rate of 35.8%; STARflo™ Glaucoma Implant CE was approved in 2012, with a 2-year effectiveness rate of 38.5%; MINIject™ was approved by CE in 2020, with a 6-month effectiveness rate of 39.1%. Drainage to the subconjunctiva includes XEN Gel Stent, which was approved by the FDA in 2016. The disadvantage is conjunctival scarring, and the 1-year effectiveness is 36.3%; PRESERFLO™ MicroShunt, which was approved by CE in 2012, has the disadvantage of conjunctival scarring, and the 5-year effectiveness is 46.7% In summary, the common shortcomings of MIGS devices at present are filter channel obstruction, fibrosis, drainage device displacement, conjunctival scarring, etc.

Taken together, different products have their own characteristics and different indications. Recently, there have been reports in foreign literature of a glaucoma drainage device operated by the external eye, which can directly drain aqueous humor to the outside of the cornea, has a significant effect in lowering intraocular pressure, and is effective in severe dry eye disease. From the perspective of the approach, external eye implant surgery is simple and convenient, and it can directly drain aqueous humor to the ocular surface, which is effective for severe dry eye. It is a kind of aqueous humor drainage device with obvious comprehensive advantages and broad application prospects.

### SUMMARY OF INVENTION

Embodiments of the present invention provide a corneal path unidirectional aqueous humor drain for solving problems in the prior art.

In order to realize the above purpose, the present invention adopts the following technical solutions.

A corneal path unidirectional aqueous drainage device, the corneal path unidirectional aqueous drainage device has a Tesla valve unit group inside the corneal path unidirectional aqueous drainage device, the Tesla valve unit group is constructed as a drainage channel; the drainage channel is a unidirectional drainage channel to drain aqueous humor from the inside of the eye to the surface of the eye.

Advantageously, the base comprises a substrate having an interconnected first side part and a second side part, and a Tesla valve unit group; the first side part has an inlet port at one end and the second side part has an outlet port at one end, and the Tesla valve unit group extends through the first side part and the second side part, and is connected to the inlet port and the outlet port; when the liquid flows into the Tesla valve unit group from the outlet port, the Tesla valve unit group is able to block the liquid from flowing to the inlet port. The Tesla valve unit is capable of blocking the flow of liquid to the inlet port when liquid flows from the outlet port.

Preferably, the Tesla valve unit group comprises a plurality of Tesla valve units connected in series with each other, each Tesla valve unit comprising a first channel and a second channel located on a radial side of the first channel; the second channel comprises a second inlet portion and a second outlet portion connected with each other, the second inlet portion and the second outlet portion each being connected to the first channel at an end, the area where the second inlet portion connects to the first channel being constructed as a The second inlet portion and the second outlet portion are each connected to the first passageway, the area where the second inlet portion is connected to the first passageway is constructed as an inlet end of the Tesla valve unit, the area where the second outlet portion is connected to the first passageway is constructed as an outlet end of the Tesla valve unit, the second inlet portion and the second outlet portion each have an angle with the first passageway, and the angle between the second inlet portion and the first passageway is greater than the angle between the second outlet portion and the first passageway, so that when a liquid enters the Tesla valve unit from the outlet end, the liquid flowing from the second inlet portion is able to block a flow of a liquid in the first passageway.

Preferably, the first passages of the adjacent Tesla valve units have mutually overlapping segments, wherein the outlet end of one Tesla valve unit and the inlet end of the other Tesla valve unit of the adjacent Tesla valve units are located on both sides of the segments; the second passages of the adjacent Tesla valve units are opposed to each other.

Preferably, the first passageway of each Tesla valve unit comprises a first inlet portion and a first outlet portion that are connected to each other; the first inlet portion and the first outlet portion of each Tesla valve unit have an angle to each other; the first inlet portion and the first outlet portion of each Tesla valve unit have an angle to each other.

Of the adjacent Tesla valve units: the first outlet portion of one Tesla valve unit overlaps with the first inlet portion of the other Tesla valve unit; the second outlet portion of one Tesla valve unit is inclined at the same angle as the first inlet portion of the other Tesla valve unit.

Preferably, the length of the base body is 1.0 mm˜6.0 mm, and the thickness is 0.05 mm˜0.5 mm; the diameters of the liquid inlet and the liquid outlet are 0.01 mm˜0.09 mm.

Preferably, the length of the base body is 3.0 mm˜4.0 mm, and the thickness is 0.15 mm˜0.3 mm; the diameters of the liquid inlet and the liquid outlet are 0.02 mm˜0.06 mm.

Preferably, the diameter of the drainage channel is 0.01 mm˜0.09 mm.

Preferably, the outer sidewall has a secondary fixation structure for preventing axial movement of the corneal path unidirectional aqueous humor drain.

Preferably, the auxiliary fixation structure comprises: a plurality of radially raised fixation portions on both radial sides of the second side portion; the plurality of fixation portions are arranged sequentially along the axial direction of the substrate, and the length of the fixation portions located on the side portion of the substrate is greater than the length of the fixation portions located in the middle portion of the substrate, so that the substrate constitutes a barbed hook-type structure.

Preferably, biocompatible materials are used.

Preferably, the biocompatible material is a light-curing biocompatible material.

Preferably, the light-curing biocompatible material comprises any one of an epoxy (meth)acrylate-based material, a polyester (meth)acrylate-based material, a polyurethane (meth)acrylate-based material, a (meth)acrylate-based monomer, and a (meth)acrylate-modified natural biomaterial.

Preferably, the photocurable biocompatible material includes triethoxybisphenol A dimethacrylate, bisphenol A epoxy methacrylate, polyethylene glycol di(meth)acrylate, (meth)acrylic acid Either ester-modified gelatin or (meth)acrylate-modified hyaluronic acid.

It can be seen from the technical solutions provided by the above embodiments of the present invention that the corneal path one-way aqueous humor drainer provided by the present invention has a Tesla valve unit group drainage channel formed in it for draining the aqueous humor to On the ocular surface, the drainage channel is one-way drainage. The aqueous humor diverter provided by the utility model introduces a Tesla valve as a structure to prevent the backflow of aqueous humor. When the liquid flows into the Tesla valve unit group from the liquid outlet, the Tesla valve unit group can block the liquid from entering the liquid. Oral flow. The aqueous humor drainage device provided by the utility model also has the following advantages: it can be used for glaucoma patients to reduce intraocular pressure and can also be used for severe dry eye; it is made of materials with good biocompatibility and has high clinical drainage efficiency; its structure It is compact, easy to model and form with 3D printing, and has the advantages of low cost and easy use.

Additional aspects and advantages of the present invention will be given in part in the following description, and these will become apparent from the following description, or by the practice of the invention.

### BRIEF DESCRIPTION OF DRAWINGS

In order to more clearly illustrate the technical solutions of the embodiments of the present invention, the following description of the embodiments of the need to use the accompanying drawings for a brief introduction, it is clear that the following description of the accompanying drawings is only some of the embodiments of the present invention, for the ordinary skilled person in the field, in the premise of not paying creative labor, but also based on the drawings of these drawings, other drawings can be obtained.

FIG. 1 shows a main view of a corneal path unidirectional aqueous humor drain provided by the present invention; the

FIG. 2 shows a sectional view of FIG. 1; the

FIG. 3 shows an elevation view of FIG. 1; the

FIG. 4 shows a left view of FIG. 1; the

FIG. 5 shows a right view of FIG. 1; the

FIG. 6 shows a schematic diagram of the structure of a Tesla valve unit group in a corneal path unidirectional aqueous humor drain provided by the present invention; the

FIG. 7 shows a schematic diagram of another preferred embodiment of a corneal path unidirectional aqueous humor drain provided by the present invention; the

FIG. 8 shows a schematic diagram of a corneal path unidirectional aqueous drainage device provided by the present invention for drainage experiments within isolated porcine ocular tissue; the

FIG. 9 shows a schematic diagram of a corneal path unidirectional aqueous drainage device provided by the present invention for safety experiments within living rabbit ocular tissue.

In the drawings, the elements represent:
1—a base, 11—a first side part, 12—a second side part, 13—an inlet port, 14—an outlet port, 15—a fixing part;
2—tesla valve unit group, 21—first passage, 211—first inlet portion, 212—first outlet portion, 22—second passage, 221—second inlet portion, 222—second outlet portion, 23—inlet end, 24—outlet end.

### DETAILED DESCRIPTION OF INVENTION

Embodiments of the present invention are described in detail below, and examples of said embodiments are shown in the accompanying drawings, wherein the same or similar symbols throughout denote the same or similar elements or elements having the same or similar functions. The embodiments described below with reference to the accompanying drawings are exemplary and are intended solely for the purpose of explaining the present invention and are not to be construed as a limitation of the invention.

It will be appreciated by those skilled in the art that the singular forms “one”, “a”, “said” and “the” used herein may also include the plural form, unless otherwise stated. “may also include the plural form. It should be further understood that the word “comprising” as used in the specification of the present invention refers to the presence of the described features, integers, steps, operations, elements and/or components, but does not exclude the presence or addition of one or more other features, integers, steps, operations, elements, components and/or groups thereof. It should be understood that when we say that a component is “connected” or “coupled” to another component, it may be directly connected or coupled to the other component, or there may be intermediate components. In addition, “connected” or “coupled” as used herein may include wirelessly connected or coupled. The term “and/or” as used herein includes any unit and all combinations of one or more associated listings.

It will be understood by those skilled in the art that all terms used herein (including technical and scientific terms) have the same meaning as generally understood by those of ordinary skill in the art to which the present invention belongs, unless otherwise defined. It should also be understood that terms such as those defined in general-purpose dictionaries are to be understood as having a meaning consistent with the meaning in the context of the prior art and are not to be construed in an idealized or overly formal sense unless defined as herein.

In order to facilitate the understanding of the embodiments of the present invention, the following will be combined with the accompanying drawings to several specific embodiments as an example of further explanation, and each embodiment does not constitute a limitation of the embodiments of the present invention.

The present invention provides a kind of aqueous humor drain for glaucoma treatment, which is used to solve the following technical problems existing in the prior art: some existing aqueous humor drainage devices are not able to effectively prevent backflow of aqueous humor, which affects the therapeutic effect; some other aqueous humor drainage devices are equipped with one-way valves, but due to the tiny volume of the device, the effect of stopping the backflow is limited, and some of the drainage devices are used to carry out the drainage through the extracorporeal device, which is inconvenient to use.

The present invention provides a corneal path unidirectional aqueous drainage device having an internal Tesla valve unit group, said Tesla valve unit group being constructed as a drainage channel which is unidirectional, i.e., capable of draining aqueous humor from the interior of the eye to the surface of the eye, and also capable of preventing the aqueous humor at the surface of the eye from flowing backward.

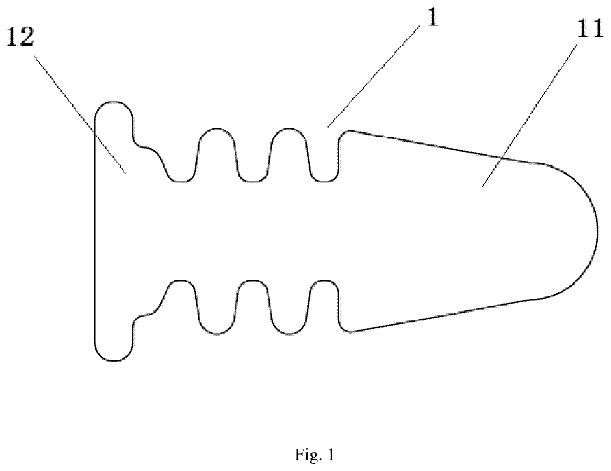

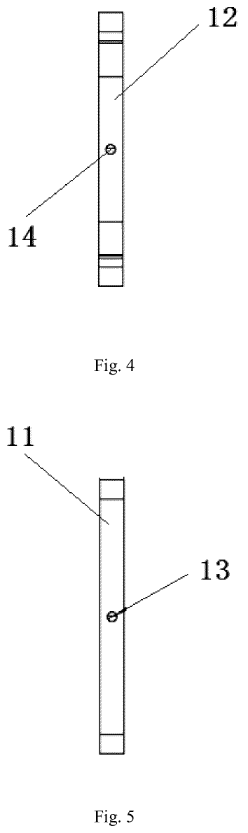

In the preferred embodiment provided by the present invention, see FIGS. 1 to 7, the house water diverter comprises a substrate 1 having a first side 11 and a second side 12 integrally molded and connected to each other, and a Tesla valve unit group 2. The house water diverter is wedged into the eye through the first side 11, which has an inlet port 13 at one end, and a discharge port 14 at the second side 12. The Tesla valve unit group 2 is used for preventing backflow of house water through the first side 11 and second side 12, and connecting the inlet port 13 and discharge port 14. The Tesla valve unit 2 extends through the first side portion 11 and the second side portion 12, and is connected to the inlet port 13 and the outlet port 14. In the embodiment provided by the present invention, the Tesla valve unit 2 is used to prevent backflow of aqueous humor (i.e., from the outlet port 14 to the inlet port 13), and the Tesla valve unit 2 is capable of blocking flow of the liquid toward the inlet port 13 when liquid flows from the outlet port 14 into the Tesla valve unit 2.

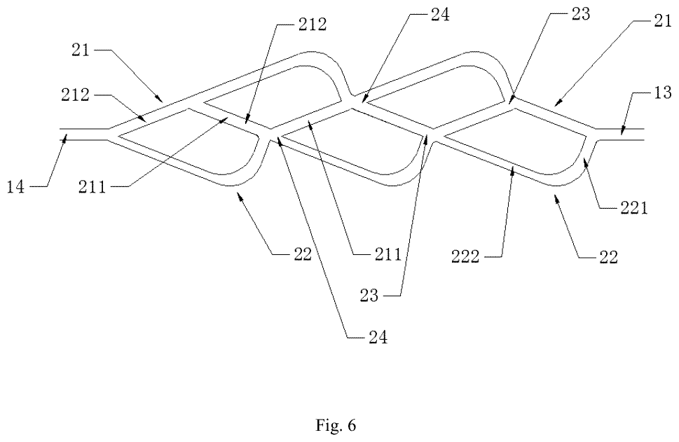

In the preferred embodiment provided by the present invention, the Tesla valve unit group 2 comprises a plurality of Tesla valve units connected in series with each other, each Tesla valve unit comprising a first passageway 21 and a second passageway 22 disposed on a radial side of the first passageway 21. The second inlet portion 221 and the second outlet portion 222 are connected to the first channel 21, respectively, the region where the second inlet portion 221 is connected to the first channel 21 (e.g., the end of both shown in FIG. 6) is constructed as an inlet end 23 of the Tesla valve unit, and the region where the second outlet portion 222 is connected to the first channel 21 is constructed as an outlet end 24 of the Tesla valve unit. The second inlet portion 221 has an angle with the first passage 21, and the angle between the second inlet portion 221 and the first passage 21 is greater than the angle between the second outlet portion 222 and the first passage 21, for example, the second inlet portion 221 and the first passage 21 are nearly perpendicular to each other. With the Tesla valve unit having the above-described setting, when liquid flows backwardly into it from the outlet end 24, a part of it flows along the first passage 21 toward the outlet end 24, and the other way flows along the second passage 22 toward the outlet end 24, and when the two liquids meet at the outlet end 24, the angle between the second inlet portion 221 and the first passage 21 is very steep, so that the liquid outflow from the second inlet portion 221 can form a larger resistance to the liquid outflow from the first passage 21, and the second inlet portion 222 is more than the first passage 21, e.g., the second inlet portion 221 is close to perpendicular to each other. When the two liquids meet at the outlet end 24, the angle between the second liquid inlet portion 221 and the first channel 21 is very steep, so that the liquid flowing out of the second liquid inlet portion 221 can form a greater resistance to the liquid flowing out of the first channel 21, blocking the flow of liquid in the first channel 21.

In order to arrange as many Tesla valve units as possible in a limited space, in some improved embodiments, as shown in FIG. 6, the first passages 21 of adjacent Tesla valve units have mutually overlapping segments (i.e., are partially overlapping). In each pair of adjacent Tesla valve units: the second passages 22 of the two Tesla valve units are arranged on radially opposite sides of the first passage 21, forming an opposing setup; the outlet end 24 of one Tesla valve unit and the inlet end 23 of the other Tesla valve unit are located at the ends of the section of the first passage 21 where the two Tesla valve units overlap with each other, respectively. Through the above setup, the second channel 22 is staggered and connected in a staggered layout, and the structure is compact and has a good space utilization rate.

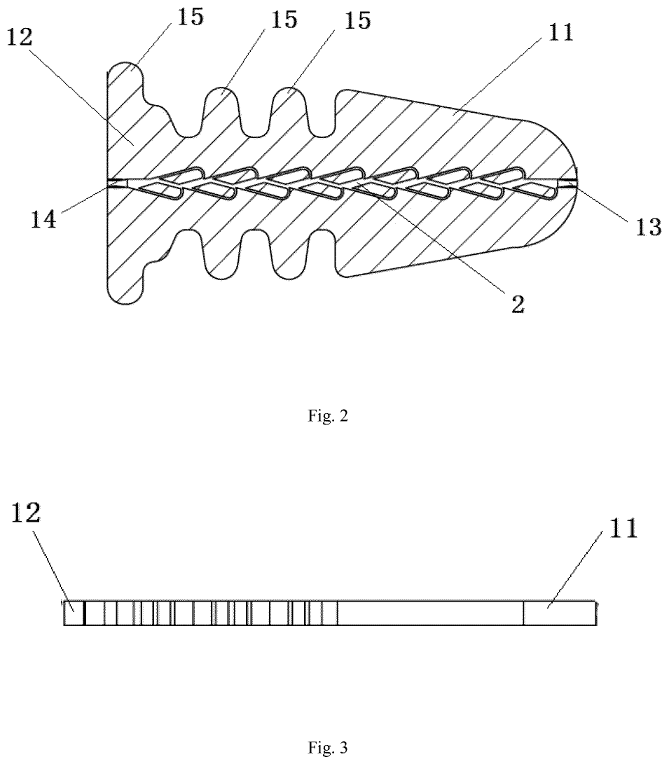

Further, the first passage 21 of each Tesla valve unit includes a first inlet portion 211 and a first outlet portion 212 that are connected to each other; the first inlet portion 211 and the first outlet portion 212 of each Tesla valve unit have an angle to each other, and when a plurality of Tesla valve units are connected in series with each other, the whole of the intermediate passageway portion composed of the first passage 21 is in the form of a folded line, as illustrated in FIGS. 2 and 6.

On this basis, in some improved embodiments, of the adjacent Tesla valve units: (based on the forward direction of the liquid from the inlet end 23 to the outlet end 24) the first outlet portion 212 of one Tesla valve unit located in the front overlaps with the first inlet portion 211 of the other Tesla valve unit located in the rear; and the angle of inclination of the second outlet portion 222 of one Tesla valve unit is the same as the angle of inclination of the first inlet portion 211 of the other Tesla valve unit. valve unit's first liquid inlet portion 211 is inclined at the same angle as the other Tesla valve unit's first liquid inlet portion 211. For example, as shown in FIGS. 2 and 6, the second outlet portion 222 of a Tesla valve unit located in the front is directly connected to the first outlet portion 212 of another Tesla valve unit located in the rear, and both of them are inclined at the same angle to form a straight channel, which is able to reduce the resistance of the forward flow of liquid, and help to discharge water from the room.

FIGS. 2 and 6 show the optimal form of a Tesla valve unit group 2: the first liquid inlet 211 and the first liquid outlet 212 of each Tesla valve unit have an included angle with each other. The absolute value of the inclination angle of the first liquid inlet part 211 and the first liquid outlet part 212 of the valve unit is the same and the direction is opposite. The middle channel part composed of the first channel 21 is in a zigzag shape as a whole; a Tesla valve located in the front The first liquid outlet 212 of the unit overlaps with the first liquid inlet 211 of another Tesla valve unit located at the rear, and the second liquid outlet 222 of the Tesla valve unit located in the front is directly connected to the other Tesla valve unit at the rear. The first liquid outlet part 212 of a Tesla valve unit has the same inclination angle, forming a linear channel as a whole; the second liquid inlet part 221 of the second channel 22 of each Tesla valve unit is connected to the second liquid outlet part 212 of the Tesla valve unit. The length of the liquid outlet part 222 is the same, and the inclination angle (direction angle) is also the same. The second liquid inlet part 221 and the first liquid inlet part 211 of each Tesla valve unit are perpendicular to each other. The connection area between the second liquid inlet part 221 and the second liquid outlet part 222 is an arc-shaped bending structure. Through the above settings, the two adjacent Tesla valve units form a mirror-symmetrical configuration with the overlapping first channel 21 as the axis, which facilitates production and processing. For example, when using a 3D printing process, everything from modeling to printing is It is more convenient and can effectively improve production efficiency.

The applicant found in the test that the axial length of the base body 1 is 1.0 mm˜6.0 mm, preferably 3.0 mm˜4.0 mm, the thickness is 0.05 mm˜0.5 mm, preferably 0.15 mm˜0.3 mm, and the liquid inlet 13 and outlet When the diameter of the liquid port 14 is 0.01 mm˜0.09 mm, preferably 0.02 mm˜0.06 mm, the finished water diversion device of this house formed using the 3D printing process can meet the requirements of structural strength and printing accuracy at the same time.

In the preferred embodiment provided by the present invention, the diameter of the drainage channel ranges from 0.01 mm to 0.09 mm, preferably from 0.02 mm to 0.06 mm.

In the preferred embodiment provided by the present invention, the present atrial water drain has an approximate barbed shape with an approximate trapezoidal configuration on one side decreasing in diameter towards its own end, the end surface of the end portion on that side having a curved surface. The other side has an auxiliary fixation structure with radial projections for preventing axial movement of said corneal path unidirectional aqueous humor drain.

Specifically, as shown in FIGS. 1 and 2, in a preferred manner, the first side portion 11 is constructed in an approximate trapezoidal shape, and the end surface of the end portion having the inlet port 13 is curved, which facilitates wedging of the drain into the eye. The auxiliary fixation structure includes: the second side portion 12 is provided with a plurality of radially raised fixation portions 15 on both radial sides thereof to prevent the drain from sliding in the eye. The plurality of fixation portions 15 are disposed sequentially along the axial direction of the substrate, and the length of the fixation portions 15 disposed on the side of the substrate 1 (i.e., adjacent to the outlet 14 of the second side portion 12) is greater than the length of the fixation portions 15 disposed in the middle of the substrate 1 (the area in which the first side portion 11 is connected to the second side portion 12). The first side portion 11 and the second side portion 12 having the above-described settings make the aqueous humor drain as a whole constitute an approximately barbed-type structure.

In another preferred embodiment, the auxiliary fixing structure is a recessed portion (not shown in the drawings) arranged in a manner similar to that of the fixing portion 15 described above, whereby adjacent recessed portions are arranged to form protrusions having an anchoring effect.

The fixing portion 15 described above is preferably a convex ring, a convex point, a convex prong, a convex tab or a threaded projection, and the recessed portion is preferably a recessed point, a recessed groove or a recessed threaded structure.

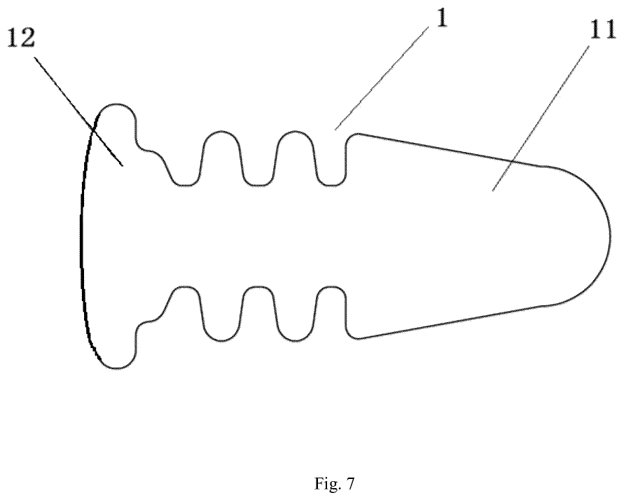

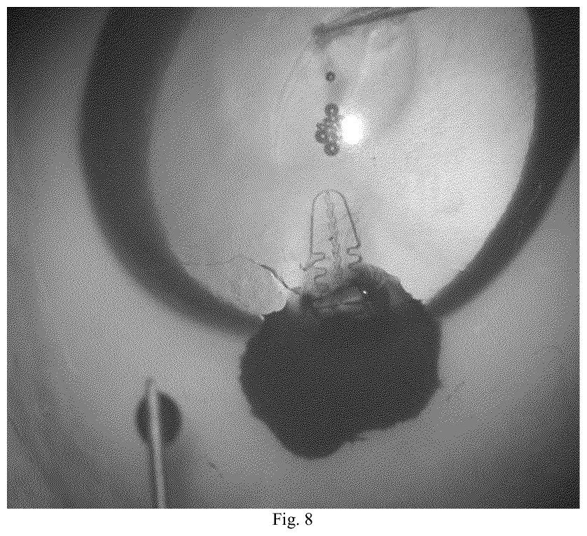

As shown in FIG. 1, this room water diverter is constructed with a curved end at one end of the axial direction and a flat surface at the other end, or it can be constructed with curved ends at both ends, as shown in FIG. 7.

In the preferred embodiment provided by the present invention, the aqueous drainage device of this invention uses biocompatible materials, preferably light-curing biocompatible materials that can be used in 3D printing processes. Specifically, they are epoxy resin (meth)acrylate materials, polyester (meth)acrylate materials, polyurethane (meth)acrylate materials, (meth)acrylate monomers and (meth)acrylic acid Any of ester-modified natural biomaterials. Preferred are triethoxylated bisphenol A dimethacrylate, bisphenol A epoxy methacrylate, polyethylene glycol di(meth)acrylate, (meth)acrylate modified gelatin, (meth)acrylate Any of acrylate-modified hyaluronic acids.

FIG. 8 shows a schematic diagram of a drainage experiment of the corneal path unidirectional aqueous drain provided by the present invention in isolated porcine ocular tissues, as can be seen, titrating the staining liquid on the side of the second lateral portion 12 of the aqueous drain wedged in the ocular tissues, and the Tesla valve unit group in the atrial aqueous drain was able to effectively prevent the staining liquid from flowing from the second lateral portion to the first lateral portion 11.

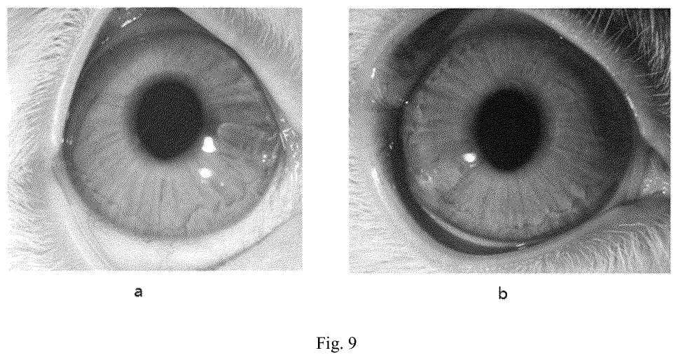

FIG. 9 is a schematic diagram of the safety experiment of the corneal path one-way aqueous humor drainage device provided by the present invention in the living rabbit eye tissue. The figure shows the state of the aqueous humor drainage device on the 4th day after being implanted in the rabbit eye tissue. The figure in the figure a is the status of rabbit No. 1, and picture b is the status of rabbit No. 2. It can be seen from the picture that there is no allergic or inflammatory reaction in the eye tissue of the two rabbits, and the safety of the aqueous humor drainage device is good.

In summary, the present invention provides a corneal path one-way aqueous humor drainage device, in which a Tesla valve unit group drainage channel is formed for draining aqueous humor to the ocular surface, and the drainage channel is a one-way drainage. The aqueous humor diverter provided by the utility model introduces a Tesla valve as a structure to prevent the backflow of aqueous humor. When the liquid flows into the Tesla valve unit group from the liquid outlet, the Tesla valve unit group can block the liquid from entering the liquid. Oral flow. The aqueous humor drainage device provided by the utility model also has the following advantages: it can be used for glaucoma patients to reduce intraocular pressure and can also be used for severe dry eye; it is made of materials with good biocompatibility and has high clinical drainage efficiency; its structure It is compact, easy to model and form with 3D printing, and has the advantages of low cost and easy use.

A person of ordinary skill in the art may understand that the accompanying drawings are only a schematic diagram of an embodiment, and the modules or processes in the accompanying drawings are not necessarily necessary for the implementation of the present invention.

From the above description of the embodiments, those skilled in the art can clearly understand that the present invention can be implemented by means of software plus a necessary general hardware platform. Based on this understanding, the technical solution of the present invention can be embodied in the form of a software product in essence or that contributes to the existing technology. The computer software product can be stored in a storage medium, such as ROM/RAM, disk, optical disk, etc., including a number of instructions to cause a computer device (which can be a personal computer, a server, or a network device, etc.) to execute the methods described in various embodiments or certain parts of the embodiments of the present invention.

Each embodiment in this specification is described in a progressive manner, and the same and similar parts of each embodiment can be referred to each other, and each embodiment focuses on the differences from other embodiments. In particular, for the device or system embodiments, since they are basically similar to the method embodiments, they are described in a simpler manner, and it is sufficient to refer to part of the description of the method embodiments for the relevant points. The above-described device and system embodiments are merely schematic, in which the unit described as a separated component may or may not be physically separated, and the component displayed as a unit may or may not be a physical unit, i.e., it may be located in a single place, or it may be distributed to a plurality of network units. Some or all of these modules can be selected according to actual needs to realize the purpose of the embodiment program. This embodiment can be understood and implemented by a person of ordinary skill in the art without creative labor.

The above mentioned, only for the invention of the better specific embodiment, but the scope of protection of the invention is not limited to this, any skilled person familiar with the technical field of the invention in the technical scope of the disclosure of the invention, can easily think of changes or replacements, should be covered by the scope of protection of the invention. Therefore, the scope of protection of the present invention should be based on the scope of protection of the claims.

What is claimed is:

1. A corneal path unidirectional atrial aqueous drainage device characterized in that said corneal path unidirectional atrial aqueous drainage device has a Tesla valve unit group inside said corneal path unidirectional atrial aqueous drainage device, said Tesla valve unit group being constructed as a drainage channel; said drainage channel being a unidirectional drainage for drainage of atrial aqueous from the eye to the ocular surface.

2. The corneal path unidirectional aqueous humor drain according to claim 1, characterized in that it comprises a substrate having a first side portion and a second side portion connected to each other, and said Tesla valve unit group; said first side portion having an inlet port at one end, and said second side port having an outlet port at one end, and said Tesla valve unit group running through said first side portion and said second side portion, and connected to said inlet port and outlet port When liquid flows into said Tesla valve unit group from said outlet port, said Tesla valve unit group is capable of blocking the flow of liquid to said inlet port.

3. The corneal path unidirectional aqueous humor drain according to claim 1, characterized in that said group of Tesla valve units comprises a plurality of Tesla valve units connected in series with each other, each of said Tesla valve units comprising a first passageway and a second passageway disposed on a radial side of said first passageway; said second passageway comprising a second inlet portion and a second outlet portion connected to each other, with one end of said second inlet portion and said second outlet portion of said second inlet portion and second outlet portion respectively, said second inlet portion being connected to said first channel, said second outlet portion being connected to said first channel being constructed as the inlet end of said Tesla valve unit, said second outlet portion being connected to said first channel being constructed as the outlet end of said Tesla valve unit, said second inlet portion and second outlet portion being angularly disposed with respect to said first channel respectively, and said second inlet portion having an angle of disposition with respect to said first channel greater than the angle of disposition between said second inlet portion and said first channel and the second outlet portion and said second outlet portion and said first channel. The angle between said second inlet portion and said first channel is greater than the angle between said second outlet portion and said first channel, so that when liquid enters said Tesla valve unit from said outlet end, liquid flowing from said second inlet portion is able to block the flow of liquid in said first channel.

4. The corneal path unidirectional aqueous humor drain according to claim 3, characterized in that said first passages of adjacent said Tesla valve units have mutually overlapping segments, with the outlet end of one of said Tesla valve units and the inlet end of the other of said Tesla valve units of said adjacent Tesla valve units being located on either side of the segment; said second passages of said Tesla valve units of said adjacent Tesla valve units being opposed to each other. said second channels of said adjacent Tesla valve units are opposed to each other.

5. The corneal path unidirectional aqueous humor drain according to claim 3, characterized in that said first channel of each said Tesla valve unit comprises a first inlet portion and a first outlet portion which are connected to each other; said first inlet portion and said first outlet portion of each said Tesla valve unit having an angle to each other;
wherein said Tesla valve units in close proximity to each other: the first outlet portion of one said Tesla valve unit overlaps with the first inlet portion of the other said Tesla valve unit; the second outlet portion of one said Tesla valve unit is inclined at the same angle as the first inlet portion of the other said Tesla valve unit.

6. The corneal path unidirectional aqueous humor drain according to claim 5, characterized in that the length of the base body is 1.0 mm˜6.0 mm, and the thickness is 0.05 mm˜0.5 mm; the liquid inlet The diameter of the mouth and liquid outlet is 0.01 mm˜0.09 mm.

7. The corneal path unidirectional aqueous humor drain according to claim 6, characterized in that the length of the base body is 3.0 mm˜4.0 mm, and the thickness is 0.15 mm˜0.3 mm; the liquid inlet and the liquid outlet The diameter of the mouth is 0.02 mm˜0.06 mm.

8. The corneal path unidirectional aqueous humor drain according to claim 5, characterized in that the diameter of the drainage channel is 0.01 mm˜0.09 mm.

9. The corneal path unidirectional aqueous humor drain according to claim 5, characterized in that the outer sidewall has an auxiliary fixation structure for preventing axial movement of said corneal path unidirectional aqueous humor drain.

10. The corneal path unidirectional aqueous humor drain according to according to claim 9, characterized in that said auxiliary fixation structure comprises: a plurality of radially projecting fixation portions on radial sides of said second side portion; said plurality of fixation portions being arranged sequentially along said axial direction of said substrate and the length of said fixation portions disposed in said side portion of said substrate being greater than the length of said fixation portions disposed in said middle portion of said substrate, so that said substrate is arranged axially along said substrate, and the length of said fixed portions at said lateral portion of said substrate is greater than the length of said fixed portions at said central portion of said substrate, so that said substrate forms a barbed structure.

11. The corneal path unidirectional aqueous humor drain according to claim 5, characterized in that a biocompatible material is used.

12. The corneal path unidirectional aqueous humor drain according to claim 11, characterized in that said biocompatible material is a light-cured biocompatible material.

13. The corneal path unidirectional aqueous humor drain according to claim 12, characterized in that said light-curing biocompatible material comprises any one of an epoxy (meth)acrylate-based material, a polyester (meth)acrylate-based material, a polyurethane (meth)acrylate-based material, a (meth)acrylate-based monomer, and a (meth)acrylate-modified natural biomaterial.

14. The corneal path unidirectional aqueous humor drain according to claim 13, characterized in that the light-curing biocompatible material includes triethoxybisphenol A dimethacrylate, bisphenol A epoxy methacrylate Any one of polyacrylate, polyethylene glycol di(meth)acrylate, (meth)acrylate-modified gelatin, and (meth)acrylate-modified hyaluronic acid.
